(19) United States
(12) Patent Application Publication
(10) Pub. No.: US 2026/0258464 A1
(43) Pub. Date: Sep. 3, 2026

(54) METHODS OF PRODUCING RNA

(71) Applicant: ModernaTX, Inc., Cambridge, MA (US)

(72) Inventors: Margaret Franklin, Cambridge, MA (US); Amy E. Rabideau, Cambridge, MA (US); Ruchi Jain, Cambridge, MA (US); Mihir Metkar, Cambridge, MA (US)

(73) Assignee: ModernaTX, Inc., Cambridge, MA (US)

(21) Appl. No.: 18/877,770

(22) PCT Filed: Jun. 23, 2023
(86) PCT No.: PCT/US2023/026043

§ 371 Date: Dec. 20, 2024

Related U.S. Application Data
(60) Provisional application No. 63/355,332, filed on Jun. 24, 2022.
(60) Provisional application No. 63/430,266, filed on Dec. 5, 2022.

(51) Int. Cl. C12P 19/34; C12N 9/12; C12N 9/14; C12N 15/113

(52) U.S. Cl. CPC C12P 19/34; C12N 9/1247; C12N 9/14; C12N 15/113; C12Y 207/07006; C12Y 306/01001; C12N 2310/315; C12N 2310/321; C12N 2310/333; C12N 2310/334; C12N 2310/335

(57) ABSTRACT

Aspects of the disclosure relate to invitro transcription reactions comprising an initiator oligonucleotide, a deoxyribonucleic acid (DNA), a ribonucleic acid (RNA) polymerase, and nucleoside triphosphates (NTPs), wherein the initiator oligonucleotide comprises at least four contiguous nucleotides. In some embodiments, the disclosure relates to new and improved compositions of initiator oligonucleotides.

### RELATED APPLICATIONS

This application claims the benefit under 35 U.S.C. § 119(e) of U.S. provisional application No. 63/355,332, filed Jun. 24, 2022, and U.S. provisional application No. 63/430,266, filed Dec. 5, 2022, each of which is incorporated by reference herein in its entirety.

### BACKGROUND

Capping of an RNA transcript increases its stability and can also prevent its degradation by exonucleases, promote its translation, and regulate its export from the nucleus (Ramanathan, A. et al. mRNA capping: biological functions and application. Nucleic Acids Res. 2016, 44(16), 7511-7526). However, the identification of new and improved strategies for further enhancement of the stability of RNA, resistance to degradation by nucleases, and promotion of its translation export regulation are desired.

### SUMMARY

Aspects of the disclosure relate to an in vitro transcription reaction comprising: an initiator oligonucleotide, a deoxyribonucleic acid (DNA), a ribonucleic acid (RNA) polymerase, and nucleoside triphosphates (NTPs), wherein the initiator oligonucleotide comprises at least four contiguous nucleotides. In some embodiments, the initiator oligonucleotide further comprises at least one synthetic modification. In some embodiments, the synthetic modification is linked to (e.g., covalently linked to) a 5′ nucleotide of the initiator oligonucleotide.

The initiator oligonucleotide may comprise at least five, at least six, or at least seven contiguous nucleotides. In some embodiments, the initiator oligonucleotide is four to ten contiguous nucleotides in length. In some embodiments, the initiator oligonucleotide consists of no more than ten nucleotides.

In some embodiments, an initiator oligonucleotide comprises contiguous nucleotides selected from adenine, guanine, and cytosine. In some embodiments, an initiator oligonucleotide comprises contiguous nucleotides consisting of a mixture of two or three nucleotides selected from adenine, guanine, and cytosine. In some embodiments, an initiator oligonucleotide lacks thymine (T). In some embodiments, an initiator oligonucleotide comprises contiguous nucleotides that consist of a heterogenous mixture of nucleotides, for example, selected from adenine, guanine, and cytosine.

In some embodiments, the NTPs comprise unmodified adenosine triphosphate (ATP), unmodified cytidine triphosphate (CTP), unmodified uridine triphosphate (UTP), and/or unmodified guanosine triphosphate (GTP). In some embodiments, the NTPs comprise modified ATP, modified CTP, modified UTP (e.g., 1-methylpseudo-UTP), and/or modified GTP.

In some embodiments, the reaction mixture comprises an equimolar concentration of NTPs. In other embodiments, the concentration of NTPs in a reaction mixture is not equimolar.

In some embodiments, the initiator oligonucleotide comprises an adenine-guanine (AG) dinucleotide. The AG dinucleotide may be at a 3′ end of the initiator oligonucleotide. In some embodiments, the initiator oligonucleotide comprises a nucleotide sequence selected from GCAAG, GGCAG, GCGAG, GCAGG, GGCGCAG, and GGCGCGCAG. In some embodiments, the initiator oligonucleotide comprises or consists of GCAAG. In some embodiments, the initiator oligonucleotide comprises or consists of GGCAG. In some embodiments, the initiator oligonucleotide comprises or consists of GCGAG. In some embodiments, the initiator oligonucleotide comprises or consists of GCAGG. In some embodiments, the initiator oligonucleotide comprises or consists of GGCGCAG. In some embodiments, the initiator oligonucleotide comprises or consists of GGCGCGCAG.

The synthetic modification may comprise, in some embodiments, a synthetic carbon chain (e.g., an alkyl chain). The reaction mixture may further comprise water, buffer, and/or pyrophosphatase. In some embodiments, the RNA polymerase is a T7 RNA polymerase. In some embodiments, the RNA polymerase is a naturally occurring RNA polymerase. In some embodiments, the reaction mixture may further comprise a ribonucleic acid (RNA) encoded by the DNA.

Some aspects describe a method comprising maintaining an in vitro transcription reaction as described herein at in vitro transcription conditions to produce a RNA. In some embodiment, the in vitro transcription conditions include maintaining a temperature of about 37 degrees Celsius for about 30 minutes to about 3 hours.

### BRIEF DESCRIPTION OF DRAWINGS

FIG. 1 provides a diagram of an in vitro transcription (IVT) reaction involving a DNA template, an RNA polymerase, and an initiator oligonucleotide (“Oligo-AG”) to produce an RNA of interest that incorporates the initiator oligonucleotide.

FIG. 2 provides a graph showing the expression of a protein encoded by an RNA of interest following (a) an in vitro transcription reaction involving a DNA template, an RNA polymerase, and an initiator oligonucleotide and (b) an in vitro translation reaction.

FIGS. 3A-3B provides graphs showing the production of an RNA of interest following an in vitro transcription (IVT) reaction involving a DNA template, an RNA polymerase, and an initiator oligonucleotide. FIG. 3A shows the percentage of RNA molecules that incorporate the initiator oligonucleotide; and FIG. 3B shows the RNA yield.

FIGS. 4A-4B provides graphs showing the production of an RNA of interest following an in vitro transcription (IVT) reaction involving a DNA template, an RNA polymerase, an initiator oligonucleotide, and varying concentrations of magnesium acetate (MgOAc). FIG. 4A shows the percentage of RNA molecules that incorporate the initiator oligonucleotide; and FIG. 4B shows the RNA yield.

FIGS. 5A-5B provides graphs showing the production of an RNA of interest following an in vitro transcription (IVT) reaction involving a DNA template, an RNA polymerase, and an initiator oligonucleotide. FIG. 5A shows the percentage of RNA molecules that incorporate the initiator oligonucleotide; and FIG. 5B shows the RNA yield over the runtime of the IVT reaction (180 minutes).

FIG. 6 provides a graph showing the percent incorporation of an initiator oligonucleotide into an RNA of interest following an in vitro transcription (IVT) reaction.

### DETAILED DESCRIPTION

Aspects of the disclosure relate to methods of producing RNA in an in vitro transcription reaction. The inventors surprisingly established effective methods of in vitro RNA transcription that do not require the presence of an RNA cap analogue (“capless transcription”). The inventors demonstrated that the methods described herein, which can enable the introduction of synthetic modifications (e.g., to the 5′ terminal end) into a transcribed RNA, are useful in slowing and/or lessening the degradation of the transcribed RNA (e.g., in vitro and following in vivo delivery of the transcribed RNA) that results from chemical and enzymatic activities (e.g., enzymatic activity of nucleases that degrade the 5′ terminal end of RNA transcripts).

Accordingly, in some aspects, the disclosure relates to an in vitro transcription (“IVT”) reaction comprising: an initiator oligonucleotide, a deoxyribonucleic acid (DNA), a ribonucleic acid (RNA) polymerase, and nucleoside triphosphates (NTPs). In some embodiments, the initiator oligonucleotide comprises at least 2, 3, 4, 5, 6, 7, or 8 contiguous nucleotides (e.g., contiguous unmodified nucleotides). In some embodiments, the initiator oligonucleotide further comprises at least one synthetic modification. IVT reaction conditions described herein typically utilize an initiator oligonucleotide, a linear DNA molecule containing a promoter, an RNA polymerase, NTPs, and a buffer system (e.g., that includes dithiothreitol (DTT) and magnesium ions) to produce an RNA of interest. The exact conditions used in any particular transcription reaction depend on the amount of RNA needed for a specific application.

### Initiator Oligonucleotide

An initiator oligonucleotide is a short nucleic acid (e.g., fewer than 20 or fewer than 10 nucleotides in length) that can be utilized to initiate (start) transcription of a DNA molecule in the presence of an RNA polymerase to produce an RNA molecule. In some embodiments, an initiator oligonucleotide is single-stranded. In other embodiments, an initiator oligonucleotide is double-stranded. In some embodiments, an initiator oligonucleotide is partially double-stranded (e.g., comprises a first nucleic acid strand bound to a second nucleic acid strand, wherein the two nucleic acid strands are not the same length or comprise the same number of nucleotides). An initiator oligonucleotide may be an RNA or DNA molecule. In some embodiments, the initiator oligonucleotide enables capless transcription (e.g., synthesis of an RNA molecule without an RNA cap analog). As used herein, the term “RNA cap analog” generally refers to a molecular entity comprising a 5′ inverted guanine nucleotide (e.g., 7-methylguanosine).

In some embodiments, an initiator oligonucleotide consists of no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, or no more than 20 nucleotides in length. In some embodiments, an initiator oligonucleotide is 3-8, 3-10, 4-10, 4-15, 4-20, 5-10, 5-15, 6-10, or 4-8 nucleotides in length. In some embodiments, an initiator oligonucleotide comprises at least 4, at least 5, at least 6, at least 7, or at least 8 nucleotides in length. In some embodiments, an initiator oligonucleotide consists of no more than ten nucleotides.

In some embodiments, an initiator oligonucleotide comprises contiguous nucleotides having a length of no more than 3 nucleotides, no more than 4 nucleotides, no more than 5 nucleotides, no more than 6 nucleotides, no more than 7 nucleotides, no more than 8 nucleotides, no more than 9 nucleotides, no more than 10 nucleotides, no more than 11 nucleotides, no more than 12 nucleotides, no more than 13 nucleotides, no more than 14 nucleotides, no more than 15 nucleotides, no more than 16 nucleotides, no more than 17 nucleotides, no more than 18 nucleotides, no more than 19 nucleotides, or no more than 20 nucleotides. In some embodiments, an initiator oligonucleotide has a length of 3-8, 3-10, 4-10, 4-15, 4-20, 5-10, 5-15, 6-10, or 4-8 nucleotides.

As is understood in the art, a nucleotide is a common unit of length for nucleic acids, for example, single-stranded nucleic acids.

In some embodiments, an initiator oligonucleotide comprises unmodified nucleotides and/or modified nucleotides. In some embodiments, each of the nucleotides of an initiator oligonucleotide is an unmodified nucleotide. In some embodiments, an initiator oligonucleotide comprises at least 3, 4, 5, 6, 7, or 8 contiguous unmodified nucleotides. In some embodiments, an initiator oligonucleotide comprises 3-8, 3-10, 4-10, 4-15, 4-20, 5-10, 5-15, or 4-8 contiguous unmodified nucleotides. In some embodiments, at least 1, 2, 3, 4, 5, 6, 7, or 8 of the nucleotides of an initiator oligonucleotide is a modified nucleotide. In some embodiments, an initiator oligonucleotide comprises 3, 4, 5, 6, 7, or 8 contiguous unmodified nucleotides and one or more additional modified nucleotides.

A modified nucleotide may comprise 2′ O-methyl nucleotide modifications, modified internucleoside bonds, and/or phosphorothioate bonds. In some embodiments, a modified nucleotide comprises phosphorylation at the 5′ terminal nucleotide. Modified nucleotides may include modified nucleobases. For example, a modified nucleobase may be selected from pseudouridine (W), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine.

An initiator oligonucleotide may comprise any sequence capable of initiating transcription of a DNA template molecule. In some embodiments, an initiator oligonucleotide comprises an adenine-guanine (AG) dinucleotide. In some embodiments, an AG dinucleotide is positioned at or near a 3′ end of the initiator oligonucleotide. For example, in some embodiments, the two nucleotides at the 3′ end of an initiator oligonucleotide are an AG dinucleotide. In some embodiments, an initiator oligonucleotide comprises a nucleotide sequence selected from GCAG, CAAG, CGAG, CAGG, GCAAG, GGCAG, GCGAG, GCAGG, GGCGCAG, and GGCGCGCAG. In some embodiments, an initiator oligonucleotide comprises or consists of GCAG. In some embodiments, an initiator oligonucleotide comprises or consists of CAAG. In some embodiments, an initiator oligonucleotide comprises or consists of CGAG. In some embodiments, an initiator oligonucleotide comprises or consists of CAGG. GCAAG. In some embodiments, an initiator oligonucleotide comprises or consists of GGCAG. In some embodiments, an initiator oligonucleotide comprises or consists of GCGAG. In some embodiments, an initiator oligonucleotide comprises or consists of GCAGG. In some embodiments, an initiator oligonucleotide comprises or consists of GGCGCAG. In some embodiments, an initiator oligonucleotide comprises or consists of GGCGCGCAG. In some embodiments, an initiator oligonucleotide comprising an AG dinucleotide comprises the nucleic acid sequence of [N]_{X1}-AG-[N]_{X2}, wherein N is any nucleotide, X₁ is a number from 1 to 20, and X₂ is a number from 0 to 2. In some embodiments, an initiator oligonucleotide comprises a nucleotide sequence selected from NNAG, NNNAG, NNNNAG, NNNNNGG, NNNNNNAG, NNNNNNNAG, and NNNNNNNNAG, wherein N is any nucleotide.

In some embodiments, an initiator oligonucleotide consists of nucleotides selected from adenine, guanine, and cytosine. In some embodiments, an initiator oligonucleotide comprises a mixture of two or three nucleotides selected from adenine, guanine, and cytosine. In some embodiments, an initiator oligonucleotide lacks thymine (T).

In some embodiments, an initiator oligonucleotide comprises or consists of RNA nucleotides. In some embodiments, an initiator oligonucleotide comprises or consists of DNA nucleotides. In some embodiments, an initiator oligonucleotide comprises a combination of RNA nucleotides and DNA nucleotides.

An initiator oligonucleotide may comprise a synthetic modification. In some embodiments, the synthetic modification is linked to a 5′ nucleotide of the initiator oligonucleotide. In other embodiments, the synthetic modification is linked to an internal nucleotide of the initiator oligonucleotide (i.e., a nucleotide that is not at the 5′ or 3′ terminal end). In some embodiments, the synthetic modification is linked to the nucleotide immediately adjacent to the 5′ terminal nucleotide. A synthetic modification may be linked to a nucleotide via a covalent linkage.

A synthetic modification may comprise a synthetic carbon chain (e.g., an alkyl chain) or an organic small molecule. In some embodiments, a synthetic modification comprises a non-nucleic acid linker molecule. In some embodiments, a synthetic modification is a synthetic chemical linker. In some embodiments, a synthetic modification comprises an alkyl carbon chain, a triethylene glycol spacer (e.g., Spacer 3, Spacer 9 or Spacer 18), a tetraethylene glycol spacer (e.g., BiotinTEG), a biotin or streptavidin molecule, an amino group, a carboxyl group, a carbonyl group, a TEG, and/or an ester. In some embodiments, a synthetic modification comprises a propane, propyl, butane, butyl, pentane, pentyl, hexane, or hexyl group. A synthetic modification may comprise a reactive group (e.g., an azide or an alkyne). In some embodiments, a reactive group may be used to enable conjugation or reaction between an agent (e.g., a small molecule) and the RNA of interest following transcription.

In some embodiments, the concentration of initiator oligonucleotide in an IVT reaction mixture is about 0.1-1 mM, 0.1-0.9 mM, 0.1-0.75 mM, 0.25-0.75 mM, 0.1-0.5 mM, 0.2-0.8 mM, 0.2-0.6 mM, 0.3-0.55 mM, 0.4-0.5 mM, or 0.5 mM. In some embodiments, the concentration of initiator oligonucleotide in an IVT reaction mixture is about 0.5-5 mM, 0.5-2 mM, 0.5-1 mM, 1-5 mM, 2-5 mM, or 1-3 mM.

In some embodiments, the molar ratio of nucleoside triphosphates to initiator oligonucleotide in the IVT reaction is greater than 1:1. For example, the molar ratio of nucleoside triphosphates to initiator oligonucleotide in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of nucleoside triphosphates to initiator oligonucleotide in the IVT reaction is between 1:1 to 100:1, 1:1 to 75:1, 1:1 to 50:1, 1:1 to 25:1, 1:1 to 20:1, 1:1 to 10:1, 1:1 to 9:1, 1:1 to 8:1, 1:1 to 7:1, 1:1 to 6:1, 1:1 to 5:1, 1:1 to 4:1, 1:1 to 3:1, 1:1 to 2:1, 2:1 to 5:1, or 5:1 to 10:1.

### DNA Encoding an RNA of Interest

The DNA encodes an RNA of interest and may be single-stranded or double-stranded. In some embodiments, the DNA is present on a plasmid or other vector. A DNA may include a polynucleotide encoding a messenger RNA (mRNA), which in turn, encodes a polypeptide of interest. A DNA, in some embodiments, includes an RNA polymerase promoter (e.g., a T7 RNA polymerase promoter) located 5′ from and operably linked to a polynucleotide encoding an mRNA and thus a polypeptide of interest. A DNA may also include a nucleotide sequence encoding a polyadenylation (polyA) tail located at the 3′ end of the polynucleotide.

In some embodiments, the DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

An RNA of interest, in some embodiments, is the product of an IVT reaction. An RNA of interest, in some embodiments, is a messenger RNA (mRNA). In some embodiments, an RNA of interest includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide.

In some embodiments, a DNA encodes one or more untranslated regions (UTRs) (sections of a nucleic acid before a start codon (5′ UTR) and after a stop codon (3′ UTR) that are not translated). In some embodiments, a nucleic acid (e.g., a ribonucleic acid (RNA), e.g., a messenger RNA (mRNA)) of the disclosure comprising an open reading frame (ORF) encoding one or more peptide epitopes further comprises one or more UTRs (e.g., a 5′ UTR or functional fragment thereof, a 3′ UTR or functional fragment thereof, or a combination thereof).

The length of the DNA, and thus the length of the RNA of interest, may vary. For example, the DNA (and/or the RNA of interest) may have a length of 200 nucleotides to 10,000 nucleotides. In some embodiments, the DNA (and/or the RNA of interest) has a length of 200-500, 200-1000, 200-1500, 200-2000, 200-2500, 200-3000, 200-3500, 200-4000, 200-4500, 200-5000, 200-5500, 200-6000, 200-6500, 200-7000, 200-7500, 200-8000, 200-8500, 200-9000, or 200-9500 nucleotides. In some embodiments, the DNA (and/or the RNA of interest) has a length of at least 200, at least 300, at least 400, at least 500, at least 600, at least 700, at least 800, at least 900, at least 1000, at least 2000, at least 3000, at last 4000, at least 5000, at least 6000, at least 7000, at least 8000, at least 9000, or at least 10,000 nucleotides.

In some embodiments, the concentration of DNA in an IVT reaction mixture is about 0.01-0.10 mg/mL, 0.01-0.09 mg/mL, 0.01-0.075 mg/mL, 0.025-0.075 mg/mL, 0.01-0.05 mg/mL, 0.02-0.08 mg/mL, 0.02-0.06 mg/mL, 0.03-0.055 mg/mL, 0.04-0.05 mg/mL, or 0.05 mg/mL.

### RNA Polymerase

Examples of RNA polymerase that may be used as provide herein include, without limitation, T7 RNA polymerase, T3 RNA polymerase, and SP6 RNA polymerase, and homologs, orthologs, and variants thereof.

In some embodiments, the RNA polymerase is a T7 polymerase variant. In some embodiments, a wild-type T7 polymerase is used in an IVT reaction. In some embodiments, a modified or mutant T7 polymerase is used in an IVT reaction. In some embodiments, a T7 RNA polymerase variant comprises an amino acid sequences that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, the RNA polymerase is a T3 polymerase variant. In some embodiments, a wild-type T3 polymerase is used in an IVT reaction. In some embodiments, a modified or mutant T3 polymerase is used in an IVT reaction. In some embodiments, a T3 RNA polymerase variant comprises an amino acid sequences that shares at least 50%, 60%, 70%, 80%, 90%, 95%, or 99% identity with a wild-type T3 (WT T3) polymerase. In some embodiments, the T3 RNA polymerase is present in a reaction (e.g., an IVT reaction) at a concentration of 0.01 mg/ml to 1 mg/ml. For example, the T3 RNA polymerase may be present in a reaction at a concentration of 0.01 mg/mL, 0.05 mg/ml, 0.1 mg/ml, 0.5 mg/ml or 1.0 mg/ml.

In some embodiments, the RNA polymerase is a SP6 polymerase variant. In some embodiments, a wild-type SP6 polymerase is used in an IVT reaction. In some embodiments, a modified or mutant SP6 polymerase is used in an IVT reaction. In some embodiments, a SP6 RNA polymerase variant comprises an amino acid sequences that shares at least 50%, 60%, 70%, 80%, 90%, 95%, or 99% identity with a wild-type SP6 (WT SP6) polymerase. In some embodiments, the SP6 RNA polymerase is present in a reaction (e.g., an IVT reaction) at a concentration of 0.01 mg/ml to 1 mg/ml. For example, the SP6 RNA polymerase may be present in a reaction at a concentration of 0.01 mg/mL, 0.05 mg/ml, 0.1 mg/ml, 0.5 mg/ml or 1.0 mg/ml.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

Global sequence alignment and local sequence alignment are two common methods used to compare and analyze sequences of DNA, RNA, or protein. Global sequence alignment compares the entire length of two sequences and finds the best possible alignment of the entire length of the sequences. It is useful, for example, when the two sequences being compared are similar in length and share significant homology. Local sequence alignment, on the other hand, identifies regions of similarity between sequences, allowing for gaps and mismatches in the alignment. This method is useful for identifying short regions of homology within larger sequences, and can be used to identify functional domains, protein families, and binding sites. Local alignment can be computationally more efficient than global alignment, and can be applied to sequences of different lengths.

Unless stated otherwise herein, “percent (%) identity” between two polynucleotides or between two proteins refers to percent (%) identity determined using a global sequence alignment, comparing the length of entire sequences (e.g., entire initiator oligonucleotide, entire polynucleotide, entire open reading frame of an mRNA, or entire protein encoded by an mRNA, as described herein).

### Nucleoside Triphosphates

NTPs of the present disclosure may be naturally-occurring NTPs, synthetic NTPs, and/or modified NTPs. A reaction mixture may include naturally-occurring NTPs, synthetic NTPs, modified NTPs, or any combination thereof. Thus, the NTPs of a reaction mixture may comprise unmodified and/or modified adenosine triphosphate (ATP), modified and/or unmodified uridine triphosphate (UTP), modified and/or unmodified guanosine triphosphate (GTP), and/or modified and/or unmodified cytidine triphosphate (CTP). In some embodiments, the NTPs include modified nucleobases. In some embodiments, the NTPs are ribonucleotide triphosphates. Non-limiting examples of modified nucleobases that may be used as provided herein include pseudouridine (p), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a mixture of NTPs (and thus the RNA transcript) includes a combination of at least two (e.g., 2, 3, 4 or more) of the foregoing modified nucleobases. In some embodiments, a mixture of NTPs comprises 1-methylpseudouridine (m1ψ). In some embodiments, a mixture of NTPs comprises 1-ethylpseudouridine.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1. The same IVT reaction may include 3.75 millimolar cap analog (e.g., trinucleotide cap or tetranucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5.

In some embodiments, the amount of NTPs in a IVT reaction is calculated empirically. For example, the rate of consumption for each NTP in an IVT reaction may be empirically determined for each individual input DNA, and then balanced ratios of NTPs based on those individual NTP consumption rates may be added to a IVT comprising multiple of the input DNAs.

In some embodiments, each individual (and thus total) NTP concentration in an IVT reaction mixture is maintained above zero (0) millimolar (mM) throughout the reaction. For example, the NTP concentrations may be maintained at 1 mM to 50 mM, 1 mM to 40 mM, 1 mM to 30 mM, 1 mM to 20 mM, 1 mM to 10 mM, 2 mM to 50 mM, 2 mM to 40 mM, 2 mM to 30 mM, 2 mM to 20 mM, 2 mM to 10 mM, 3 mM to 50 mM, 3 mM to 40 mM, 3 mM to 30 mM, 3 mM to 20 mM, 3 mM to 10 mM, 4 mM to 50 mM, 4 mM to 40 mM, 4 mM to 30 mM, 4 mM to 20 mM, 4 mM to 10 mM, 5 mM to 50 mM, 5 mM to 40 mM, 5 mM to 30 mM, 5 mM to 20 mM, or 5 mM to 10 mM. In some embodiments, the NTP concentrations are maintained at 10 mM to 20 mM. In some embodiments, the NTP concentrations are maintained at (or at least at) 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some embodiments, the NTP concentrations are maintained above a lower limit of 2 mM to 10 mM, above a lower limit of 2 mM to 9 mM, above a lower limit of 2 mM to 8 mM, above a lower limit of 2 mM to 7 mM, above a lower limit of 2 mM to 6 mM, or above a lower limit of 2 mM to 6 mM.

The relative concentration of individual NTPs in an IVT reaction may also vary. For example, ATP may be used in excess of GTP, CTP and UTP. As other examples, GTP may be used in excess of ATP, CTP and UTP, CTP may be used in excess of ATP, GTP and UTP, or UTP may be used in excess of ATP, GTP and CTP.

### Additional IVT Reaction Components

In some embodiments, the IVT reaction mixture comprises a buffer, e.g., Tris, phosphate or a Good's buffer. The concentration of buffer used in an IVT reaction mixture may be, for example, at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM. In some embodiments, the buffer comprises Tris-HCl. For example, the buffer may comprise 10-100 mM, 10-80 mM, 10-60 mM, 20-100 mM, 20-18 mM, 20-60 mM Tris-HCl. In some embodiments, the buffer comprises 40 mM Tris-HCl.

The buffer system of an IVT reaction mixture may vary. In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, the IVT reaction mixture contains a reductant or reducing agent such as dithiothreitol (DTT) or tris(2-carboxyethyl)phosphine (TCEP). The concentration of DTT used in an IVT reaction mixture may be, for example, at least 1 mM, at least 5 mM, or at least 50 mM. In some embodiments, the concentration of DTT used in an IVT reaction mixture is 1-50 mM or 5-50 mM. In some embodiments, the concentration of DTT used in an IVT reaction mixture is 5 mM. The concentration of TCEP used in an IVT reaction mixture may be, for example, at least 1 mM, at least 5 mM, or at least 50 mM. In some embodiments, the concentration of TCEP used in an IVT reaction mixture is 1-50 mM or 5-50 mM. In some embodiments, the concentration of TCEP used in an IVT reaction mixture is 2 mM.

In some embodiments, the IVT reaction mixture comprises magnesium. In some embodiments, the molar ratio of NTP to magnesium ions (Mg²⁺; e.g., Mg(OAc)₂) present in an IVT reaction is 1:1 to 1:5. For example, the molar ratio of NTP to magnesium ions may be 1:1, 1:2, 1:3, 1:4 or 1:5. In some embodiments, the IVT reaction mixture comprises 10-100 mM magnesium (e.g., magnesium acetate), 10-75 mM magnesium, 10-50 mM magnesium, or 10-25 mM magnesium. In some embodiments, the IVT reaction mixture comprises 10 mM magnesium, 15 mM magnesium, 20 mM magnesium, 25 mM magnesium, 30 mM magnesium, 35 mM magnesium, 40 mM magnesium, 45 mM magnesium, 50 mM magnesium, 55 mM magnesium, or 60 mM magnesium. In some embodiments, the IVT reaction mixture comprises 30-60 mM magnesium.

### Methods of Producing RNA

Some aspects relate to methods of producing (e.g., synthesizing) a RNA transcript (e.g., mRNA transcript) comprising maintaining an in vitro transcription reaction as described herein (e.g., an in vitro transcription reaction comprising an initiator oligonucleotide, a deoxyribonucleic acid, a ribonucleic acid polymerase, and nucleoside triphosphates) at in vitro transcription conditions to produce the RNA transcript. In vitro transcription conditions may, in some embodiments, include maintaining a temperature of about 37 degrees Celsius for about 30 minutes to about 3 hours.

In some embodiments, maintaining an in vitro transcription reaction at in vitro transcription conditions to produce an RNA transcript comprises maintaining a temperature of 20 degrees Celsius to 50 degrees Celsius, 30 to 40 degrees Celsius, or 35 to 40 degrees Celsius. In some embodiments, maintaining an in vitro transcription reaction at in vitro transcription conditions to produce an RNA transcript comprises maintaining a constant temperature for at least 15, at least 30, at least 60, at least 90, or at least 120 minutes. In some embodiments, maintaining an in vitro transcription reaction at in vitro transcription conditions to produce an RNA transcript comprises maintaining a constant temperature for 15-240 minutes, 30-240 minutes, 30-180 minutes, 30-120 minutes, 30-60 minutes, 60-180 minutes, or 60-120 minutes. In some embodiments, maintaining an in vitro transcription reaction at in vitro transcription conditions to produce an RNA transcript comprises maintaining a constant temperature for 1-4 hours, 1-3 hours, or 1-2 hours.

In some embodiments, IVT methods further comprise a step of separating (e.g., purifying) in vitro transcription products (e.g., mRNA) from other reaction components. In some embodiments, the separating comprises performing chromatography on the IVT reaction mixture. In some embodiments, the chromatography comprises size-based (e.g., length-based) chromatography. In some embodiments, the chromatography comprises oligo-dT chromatography.

### PolyA Tails

Aspects of the disclosure relate to methods of producing RNA compositions that have increased purity (e.g., as measured by the percentage of mRNAs comprising polyA tails) than previous IVT methods. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail.

In some embodiments, terminal groups on the poly A tail can be incorporated for stabilization. Polynucleotides can include des-3′ hydroxyl tails. They can also include structural moieties or 2′-O methyl modifications as taught by Junjie Li, et al. (Current Biology, Vol. 15, 1501-1507, Aug. 23, 2005, the contents of which are incorporated herein by reference in its entirety for this purpose).

Unique polyA tail lengths provide certain advantages to nucleic acids. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA binding protein can enhance expression.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/66242.

In some embodiments, the lipid nanoparticle comprises at least one ionizable amino lipid, at least one non-cationic lipid, at least one sterol, and/or at least one polyethylene glycol (PEG)-modified lipid. In some embodiments, the lipid nanoparticle comprises a molar ratio of 20-60% ionizable amino lipid, 5-25% non-cationic lipid, 25-55% structural lipid, and 0.5-15% PEG-modified lipid. In some embodiments, the lipid nanoparticle comprises a molar ratio of 20-60% ionizable amino lipid, 5-30% non-cationic lipid, 10-55% structural lipid, and 0.5-15% PEG-modified lipid. In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid. In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Pharmaceutical Compositions

Some aspects relate to pharmaceutical compositions comprising RNA compositions produced by methods described by the disclosure. RNA compositions described herein may be formulated or administered in combination with one or more pharmaceutically-acceptable excipients. As a non-limiting set of examples, RNA compositions can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with RNA compositions (e.g., for transplantation into a subject), hyaluronidase, nanoparticle mimics and combinations thereof.

In some embodiments, RNA compositions comprise at least one additional active substance, such as, for example, a therapeutically-active substance, a prophylactically-active substance, or a combination of both. RNA compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety for this purpose).

Formulations of the RNA compositions described herein may be prepared by any method known or hereafter developed in the art of pharmacology. In general, such preparatory methods include the step of bringing the active ingredient(s) (e.g., mRNAs of the composition) into association with an excipient and/or one or more other accessory ingredients, and then, if necessary and/or desirable, dividing, shaping and/or packaging the product into a desired single- or multi-dose unit.

The formulation of any of the compositions disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

In some embodiments, the compositions described herein may be formulated as lipid nanoparticles (LNPs). Accordingly, the present disclosure also relates to nanoparticle compositions comprising (i) a lipid composition comprising a delivery agent, and (ii) an RNA composition. In such nanoparticle composition, the lipid composition disclosed herein can encapsulate the nucleic acid encoding one or more peptide epitopes.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos.: WO2017075531, WO2015199952, WO2013086354, or WO2013116126, or selected from formulae CLI-CLXXXXII of U.S. Pat. No. 7,404,969.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In some embodiments, the ionizable amino lipid may have a positively charged hydrophilic head and a hydrophobic tail that are connected via a linker structure. In addition to these, an ionizable lipid may also be a lipid including a cyclic amine group.

RNA compositions can be formulated into lipid nanoparticles. In some embodiments, the lipid nanoparticle comprises at least one ionizable amino lipid, at least one non-cationic lipid, at least one sterol, and/or at least one polyethylene glycol (PEG)-modified lipid. Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle composition.

### Applications

RNA compositions produced by the methods described herein typically comprise a synthetic modification (e.g., linked to the 5′ nucleotide of the RNA of interest produced by the IVT reaction). The presence of the synthetic modification may, in some embodiments, cause the RNA of interest to have (1) a longer half-life in vitro and/or in vivo; (2) higher resistance to degradation (e.g., from nucleases); and/or (3) increased stability, relative to a control RNA that does not comprise a synthetic modification. The RNA of interest may be an mRNA (e.g., modified mRNA or unmodified RNA), a lncRNA, a self-replicating RNA, a circular RNA, a CRISPR guide RNA, or any other RNA. In embodiments, the RNA is an mRNA. In embodiments, the RNA is an unmodified mRNA. In embodiments, the RNA is a chemically modified mRNA, for example, the mRNA comprises methylpseudouridine. In embodiments, the RNA is RNA (e.g., mRNA or self-replicating RNA) that encodes a peptide or polypeptide (e.g., a therapeutic peptide or therapeutic polypeptide). The RNA transcripts produced using RNA polymerase variants may be used in a myriad of applications.

Therapeutic proteins mediate a variety of effects in a host cell or in a subject to treat or prevent a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders. Other diseases and conditions are encompassed herein.

In some embodiments, a therapeutic peptide or therapeutic protein is a biologic. A biologic is a polypeptide-based molecule that may be used to treat, cure, mitigate, prevent, or diagnose a serious or life-threatening disease or medical condition. Biologics include, but are not limited to, allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others.

In some embodiments, the therapeutic protein is a cytokine, a growth factor, an antibody (e.g., monoclonal antibody), a fusion protein, or a vaccine (e.g., a collection of RNAs encoding peptide antigens designed to elicit an immune response in a subject). Non-limiting examples of therapeutic proteins include blood factors (such as Factor VIII and Factor VII), complement factors, Low Density Lipoprotein Receptor (LDLR) and MUT1. Non-limiting examples of cytokines include interleukins, interferons, chemokines, lymphokines and the like. Non-limiting examples of growth factors include erythropoietin, EGFs, PDGFs, FGFs, TGFs, IGFs, TNFs, CSFs, MCSFs, GMCSFs and the like. Non-limiting examples of antibodies include adalimumab, infliximab, rituximab, ipilimumab, tocilizumab, canakinumab, itolizumab, tralokinumab, anti-influenza virus monoclonal antibody, anti-Chikungunya virus monoclonal antibody, anti-Zika virus monoclonal antibody, anti-SARS-CoV-2 monoclonal antibody. Non-limiting examples of fusion proteins include, for example, etanercept, abatacept and belatacept. Non-limiting examples of vaccines include, for example, Cytomegalovirus (CMV) vaccine, and personalized cancer vaccines.

One or more biologics currently being marketed or in development may be encoded by the RNA. While not wishing to be bound by theory, it is believed that incorporation of the encoding polynucleotides of a known biologic into the RNA described herein will result in improved therapeutic efficacy due at least in part to the specificity, purity and/or selectivity of the construct designs.

An RNA composition as disclosed herein may encode one or more antibodies (e.g., may comprise a first mRNA encoding an antibody heavy chain and a second RNA encoding an antibody light chain). The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. A monoclonal antibody is an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site.

Monoclonal antibodies specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies include, but are not limited to, “primatized” antibodies comprising variable domain antigen-binding sequences derived from a non-human primate (e.g., Old World Monkey, Ape etc.) and human constant region sequences.

Antibodies encoded in the RNA compositions may be utilized to treat conditions or diseases in many therapeutic areas such as, but not limited to, blood, cardiovascular, CNS, poisoning (including antivenoms), dermatology, endocrinology, gastrointestinal, medical imaging, musculoskeletal, oncology, immunology, respiratory, sensory and anti-infective.

An RNA composition as disclosed herein may encode one or more vaccine antigens. A vaccine antigen is a biological preparation that improves immunity to a particular disease or infectious agent. One or more vaccine antigens currently being marketed or in development may be encoded by the RNA. Vaccine antigens encoded in the RNA may be utilized to treat conditions or diseases in many therapeutic areas such as, but not limited to, cancer, allergy and infectious disease. In some embodiments, a vaccine may be a personalized vaccine in the form of a concatemer or individual RNAs encoding peptide epitopes or a combination thereof.

An RNA composition as disclosed herein may be designed to encode on or more antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals. The anti-microbial polypeptides may block cell fusion and/or viral entry by one or more enveloped viruses (e.g., HIV, HCV). For example, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the transmembrane subunit of a viral envelope protein, e.g., HIV-1 gp120 or gp41. The amino acid and nucleotide sequences of HIV-1 gp120 or gp41 are described in, e.g., Kuiken et al., (2008). “HIV Sequence Compendium,” Los Alamos National Laboratory.

In some embodiments, RNA transcripts are used as radiolabeled RNA probes. In some embodiments, RNA transcripts are used for non-isotopic RNA labeling. In some embodiments, RNA transcripts are used as guide RNA (gRNA) for gene targeting. In some embodiments, RNA transcripts (e.g., mRNA) are used for in vitro translation and micro injection. In some embodiments, RNA transcripts are used for RNA structure, processing and catalysis studies. In some embodiments, RNA transcripts are used for RNA amplification. In some embodiments, RNA transcripts are used as anti-sense RNA for gene expression experiment. Other applications are encompassed by the present disclosure.

### EXAMPLES

### Example 1. Initiator Oligonucleotides can be Used to Initiate Transcription

In vitro transcription reactions were performed to determine whether initiator oligonucleotides could be used to initiate (start) transcription of a DNA template. Reactions were performed at 37° C. for 120 min using a one-pot setup with 2000 ng A-start DNA template, 3 mM ATP, 3 mM GTP, 3 mM CTP, 3 mM UTP/N1-Methylpseudo-UTP, 0.05 mg/mL T7 RNA polymerase variant, and an IVT buffer. Initiator oligonucleotides as described in Table 1 were included in the reactions at 0.5 mM concentrations. Control experiments utilized GpppGAG cap analog (positive control) and the absence of an initiator oligonucleotide or cap analog (negative control).

Following the IVT reactions, each reaction was analyzed using on LC/MS to determine percent incorporation of the initiator oligonucleotides. Initiator oligonucleotides comprising five nucleotides with an AG dinucleotide at their 3′ terminal ends were capable of producing RNA transcripts with high levels of incorporation of the initiator oligonucleotide (Experiments 1-3). Additionally, an initiator oligonucleotide comprising five nucleotides but lacking a 3′ terminal AG dinucleotide was also capable of producing RNA transcripts with incorporated initiator oligonucleotide (Experiment 6). Furthermore, initiator oligonucleotides comprising the nucleic acid sequence of GGCAG and comprising a synthetic modification (a triethylene glycol spacer—Spacer 3, or a tetraethylene glycol spacer—BiotinTEG) covalently linked to the 5′ terminal nucleotide was capable of producing RNA transcripts with incorporation of the initiator oligonucleotide (Experiments 4 and 5).

TABLE 1

| | Initiator | % |
|---|---|---|
| Experiment No. | oligonucleotide | incorporation |
| Positive control | None | 90.9 |
| (GpppGAG cap analog) | | |
| Experiment 1 | GCAAG | 76.3 |
| Experiment 2 | GGCAG | 75.7 |
| Experiment 3 | GCGAG | 55.6 |
| Experiment 4 | Spacer3-GGCAG | 50.0 |
| Experiment 5 | BiotinTEG-GGCAG | 24.1 |
| Experiment 6 | GCAGG | 19.7 |
| Negative control | None | 0.0 |

### Example 2. Initiator Oligonucleotides can be Used to Initiate Transcription

In vitro transcription reactions were performed to determine the impact of including various synthetic modifications and modified nucleotides into an initiator oligonucleotide comprising a length of five nucleotides. Reactions were performed at 37° C. for 120 min using a one-pot setup with 2000 ng A-start DNA template (encoding a fluorescent protein), 3 mM ATP, 3 mM GTP, 3 mM CTP, 3 mM UTP/N1-Methylpseudo-UTP, 0.05 mg/mL T7 RNA polymerase variant, and an IVT buffer. Initiator oligonucleotides as described in Table 2 were included in the reactions at 0.5 mM concentrations. Control experiments utilized GpppGAG cap analog (positive control) and the absence of an initiator oligonucleotide or cap analog (negative control). Following the IVT reactions, each reaction was analyzed using on LC/MS to determine percent incorporation of the initiator oligonucleotides.

Each of the initiator oligonucleotides used in this Example comprised a base nucleotide sequence of GGAAG. The tested initiator oligonucleotides varied in their inclusion of types of nucleotide modifications (presence of 2′ O-methyl nucleotides (2′ Ome nucleotides), phosphorothioate internucleoside bonds (PS bonds), and/or phosphorylation at 5′ end) and synthetic modifications at the 5′ end. The initiator oligonucleotide comprising an unmodified GGAAG was capable of producing RNA transcripts with 100% incorporated initiator oligonucleotide. Inclusion of up to three 2′ O-methyl nucleotides (Experiments 2-3) or up to three phosphorothioate internucleoside bonds (Experiments 4-6) into the initiator oligonucleotide also produced RNA transcripts with 100% incorporated initiator oligonucleotide. Inclusion of three 2′ O-methyl nucleotides and three phosphorothioate internucleoside bonds (Experiment 7), or inclusion of phosphorylation at 5′ end (Experiment 8) into the initiator oligonucleotide produced lower levels of RNA transcripts with incorporated initiator oligonucleotide. However, inclusion of three 2′ O-methyl nucleotides, three phosphorothioate internucleoside bonds, and phosphorylation at 5′ end (Experiment 9) performed better than separation of those modifications (RNA transcripts with 89.7% incorporated initiator oligonucleotide). Additional experiments demonstrated that synthetic modifications (e.g., Biotin) could be added to the 5′ end of an initiator oligonucleotide comprising unmodified nucleotides and produce high levels of RNA transcripts with incorporated initiator oligonucleotide (Experiment 10); and that synthetic modifications (e.g., Spacer 18) could be added to the 5′ end of an initiator oligonucleotide comprising three 2′ O-methyl nucleotides and three phosphorothioate internucleoside bonds.

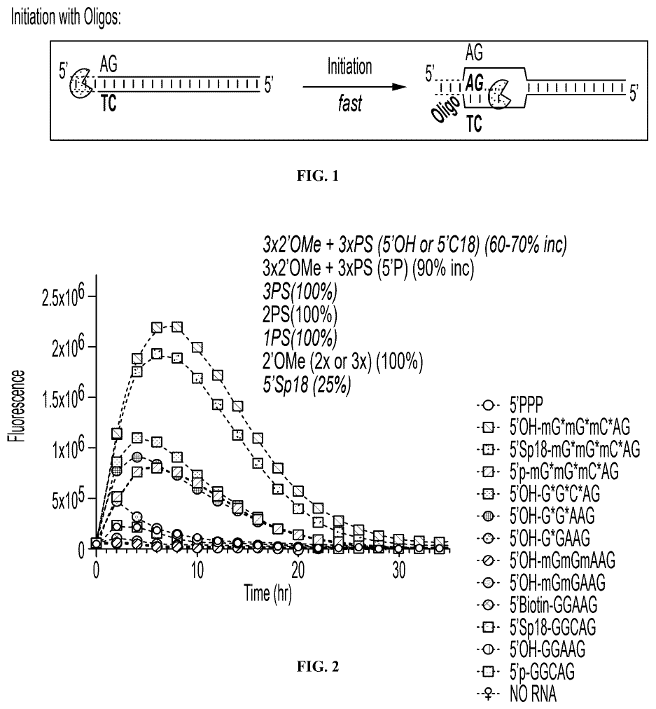

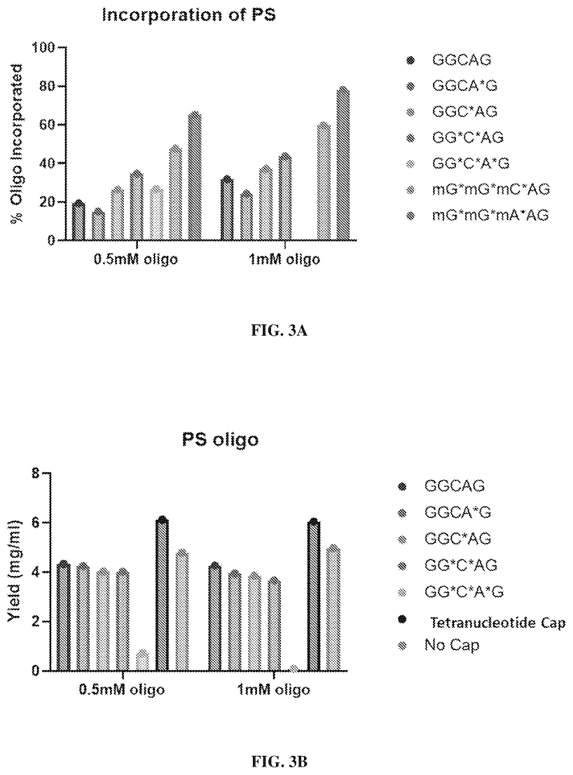

An increased number of phosphorothioate bonds within the initiator oligonucleotides led to higher incorporation of the initiator oligonucleotide into the RNA (FIG. 3A). This observation was consistent in reactions that utilized 1 mM concentrations of initiator oligonucleotides. The produced mRNA for each experiment was subjected to an in vitro translation reaction for up to 35 hours. The mRNA was added to HEK293-L2K cells and protein expression of the fluorescent protein was observed based on fluorescence. The presence of nucleotide modifications within the initiator oligonucleotide of the mRNA provided increased protein expression relative to mRNA comprising an unmodified initiator oligonucleotide. Specifically, inclusion of 2′ O-methyl nucleotides (2′ Ome nucleotides) and/or phosphorothioate internucleoside bonds (PS bonds) within the initiator oligonucleotide provided high levels of protein expression after two hours post-introduction to the HEK293-L2K cells (FIG. 2).

TABLE 2

| | Initiator | | | Number of | Number | Protein |
|---|---|---|---|---|---|---|
| Experiment | oligo- | % | Modification | 2′Ome | of PS | Expression |
| No. | nucleotide | incorporation | at 5′end | nucleotides | bonds | Detected? |
| Experiment 1 | GGAAG | 100 | OH | 0 | 0 | No |
| Experiment 2 | mGmGAAG | 100 | OH | 2 | 0 | No |
| Experiment 3 | mGmGmAAG | 100 | OH | 3 | 0 | No |
| Experiment 4 | G*GAAG | 100 | OH | 0 | 1 | Yes |
| Experiment 5 | G*G*AAG | 100 | OH | 0 | 2 | Yes |
| Experiment 6 | G*G*C*AG | 100 | OH | 0 | 3 | Yes |
| Experiment 7 | mG*mG*mC*AG | 55.4 | OH | 3 | 3 | Yes |
| Experiment 8 | pGGCAG | 47.7 | P | 0 | 0 | No |
| Experiment 9 | pmG*mG*mC*AG | 89.7 | P | 3 | 3 | Yes |
| Experiment 10 | Biotin-GGAAG | 71.2 | Biotin | 0 | 0 | Yes |
| Experiment 11 | Sp18-GGCAG | 25.5 | Spacer 18 | 0 | 0 | Yes |
| Experiment 12 | Sp18- | 67.7 | Spacer 18 | 3 | 3 | Yes |
| | mG*mG*mC*AG | | | | | |
| Positive | None | 100 | Cap analog | 0 | 0 | Yes |
| control | | | | | | |
| (GpppGAG | | | | | | |
| cap analog) | | | | | | |
| Negative | None | 0 | PPP | 0 | 0 | Yes |
| control | | | | | | |

m: 2′ O-methyl nucleotide; *: phosphorothioate (PS) bond; p: 5′ phosphorylation; Sp18: 5′ Spacer 18

### Example 3. Effects of Salt Concentrations on RNA Production

In vitro transcription reactions were performed to determine the impact of increasing salt concentrations (magnesium acetate (MgOAc)) on RNA production in the presence of modified initiator oligonucleotide comprising a length of five nucleotides. Reactions were performed at 37° C. for 120 minutes using a one-pot setup with 2000 ng A-start DNA template (encoding a fluorescent protein), 3 mM ATP, 3 mM GTP, 3 mM CTP, 3 mM UTP/N1-Methylpseudo-UTP, 0.05 mg/mL T7 RNA polymerase variant, an IVT buffer, and varying concentrations of magnesium acetate (30 mM, 40 mM, 50 mM). Initiator oligonucleotides (GG*C*A*G; mGmG*mC*A*G; mGmG*mA*A*G) were included in the reactions at 0.5 mM concentrations. Control experiments utilized a tetranucleotide cap. Following the IVT reactions, each reaction was analyzed using on LC/MS to determine percent incorporation of the initiator oligonucleotides and yield.

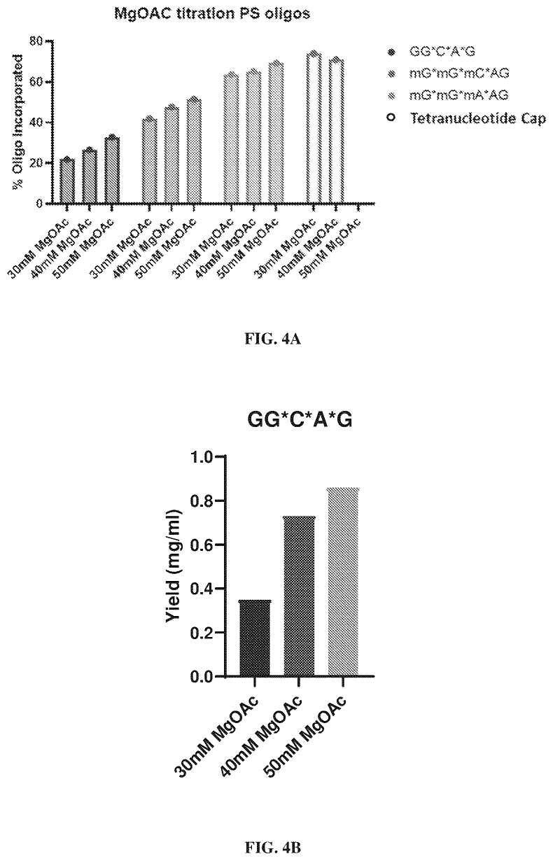

Increasing concentrations of magnesium acetate led to increasing incorporation of the initiator oligonucleotides within the produced RNA (FIG. 4A). Furthermore, increasing concentrations of magnesium acetate was able to provide increased yield of total RNA (FIG. 4B).

### Example 4. Initiator Oligonucleotides can be Used to Initiate Transcription

In vitro transcription reactions were performed to initiate (start) transcription of a DNA template using an initiator oligonucleotide. Reactions were performed at 37° C. for 120 min using a one-pot setup with 2000 ng A-start DNA template, 3 mM ATP, 3 mM GTP, 3 mM CTP, 3 mM UTP/N1-Methylpseudo-UTP, 0.05 mg/mL T7 RNA polymerase variant, and an IVT buffer. One initiator oligonucleotide—GCGAG, GGCAG, GCAAG, GCAGG, spacer3-GGCAG, G-spacer3-GCAG (“Gc3GCAG”), or GCAG—was included in each reaction at 0.5 mM concentrations. Control experiments utilized a tetranucleotide cap (positive control); and the absence of an initiator oligonucleotide or cap analog (negative control).

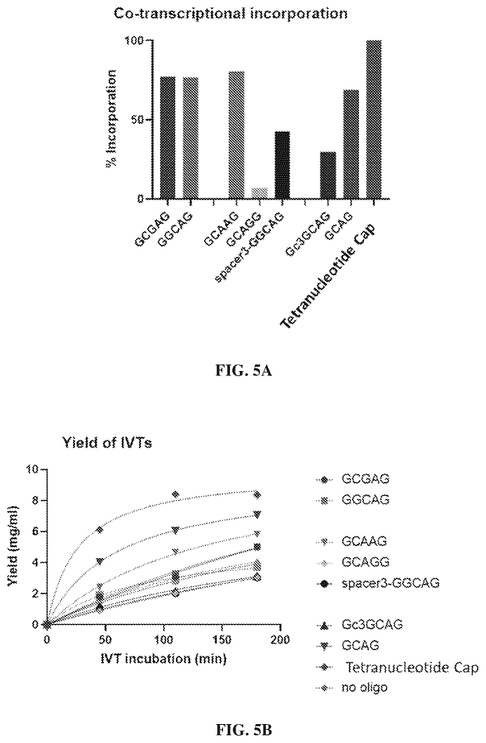

Following the IVT reactions, each reaction was analyzed using on LC/MS to determine percent incorporation of the initiator oligonucleotides. Several initiator oligonucleotides comprising four or five nucleotides with an AG dinucleotide at their 3′ terminal ends (GCGAG, GGCAG, GCAAG, GCAG) were capable of producing RNA transcripts with high levels of incorporation (e.g., over 60% incorporation) of the initiator oligonucleotide (FIG. 5A). Additionally, initiator oligonucleotides comprising a nucleic acid sequence and a synthetic modification covalently linked to the 5′ terminal nucleotide (spacer3-GGCAG, G-spacer3-GCAG) were capable of producing RNA transcripts with incorporation of the initiator oligonucleotide (FIG. 5A). Each of the tested initiator oligonucleotides produced RNA yields that were comparable to experiments performed without any initiator oligonucleotide (i.e., negative control), indicating that the presence of the initiator oligonucleotides did not negatively impact RNA yield and production in these IVT reactions.

### Example 5. Initiator Oligonucleotides with Complex Modification Patterns can be Used to Initiate Transcription

In vitro transcription reactions were performed to initiate (start) transcription of a DNA template using an initiator oligonucleotide. Reactions were performed at 37° C. for 120 min using a one-pot setup with 2000 ng A-start DNA template, 3 mM ATP, 3 mM GTP, 3 mM CTP, 3 mM UTP/N1-Methylpseudo-UTP, 0.05 mg/mL T7 RNA polymerase variant, 0.5 mM concentration of initiator oligonucleotide (or a tetranucleotide cap as a positive control), and an IVT buffer. The initiator oligonucleotides that were utilized for this Example are provided in Table 3.

TABLE 3

| | Initiator | |
|---|---|---|
| Experiment No. | oligonucleotide | Modification Status |
| Experiment 1 | pGGCAG | 5′ phosphorus |
| Experiment 2 | mGmGmCAG | 3x 2′Ome nucleotides |
| Experiment 3 | G*G*C*AG | 3x phosphorothioate bonds |
| Experiment 4 | spacer18GGCAG | 5′ Spacer 18 |
| Experiment 5 | mG*mG*mC*AG | 3x 2′Ome nucleotides, 3x |
| | | phosphorothioate bonds |
| Experiment 6 | pmG*mG*mC*AG | 5′ phosphorus, 3x 2′Ome nucleotides, |
| | | 3x phosphorothioate bonds |
| Experiment 7 | spacer18mG*mG*mC*AG | 5′ Spacer 18, 3x 2′Ome, 3x |
| | | phosphorothioate bonds |
| Experiment 8 | spacer18GGCAG | 5′ Spacer 18 |
| Experiment 9 | GGCGAG | unmodified |
| Positive control | Tetranucleotide cap | |

m: 2′ O-methyl nucleotide; *: phosphorothioate (PS) bond; p: 5′ phosphorylation; Sp18: 5′ Spacer 18

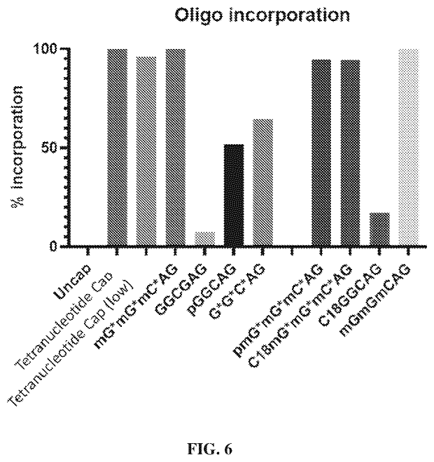

Following the IVT reactions, each reaction was analyzed using on LC/MS to determine percent incorporation of the initiator oligonucleotides. As shown in FIG. 6, the tested initiator oligonucleotides were successfully incorporated into RNA produced in IVT reactions for this Example. It was found that incorporation of initiator oligonucleotides was possible for a variety of modification patterns including combinations of 2′ O-methyl nucleotides (2′ Ome nucleotides) and phosphorothioate internucleoside bonds (PS bonds); and oligonucleotide lengths. For example, it was found that an initiator oligonucleotide comprising six nucleotides (Experiment 9) was capable of being incorporated into RNA produced in an IVT reaction.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the specific subject matter for which each is cited.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An in vitro transcription reaction comprising: an initiator oligonucleotide, a deoxyribonucleic acid (DNA), a ribonucleic acid (RNA) polymerase, and nucleoside triphosphates (NTPs), wherein the initiator oligonucleotide comprises at least four contiguous nucleotides.

2. The in vitro transcription reaction of claim 1, wherein the initiator oligonucleotide further comprises at least one synthetic modification.

3. The in vitro transcription reaction of claim 2, wherein the synthetic modification is linked to a 5′ nucleotide of the initiator oligonucleotide, optionally covalently linked to a 5′ nucleotide of the initiator oligonucleotide.

4. The in vitro transcription reaction of any one of the preceding claims, wherein the initiator oligonucleotide comprises at least five, at least six, or at least seven contiguous nucleotides.

5. The in vitro transcription reaction of claim 4, wherein the initiator oligonucleotide is four to ten contiguous nucleotides in length.

6. The in vitro transcription reaction of any one of the preceding claims, wherein the initiator oligonucleotide consist of no more than ten nucleotides.

7. The in vitro transcription reaction of any one of the preceding claims, wherein the initiator oligonucleotide comprises modified nucleotides.

8. The in vitro transcription reaction of any one of the preceding claims, wherein the initiator oligonucleotide comprises 2′-O-methyl nucleotides and/or phosphorothioate bonds.

9. The in vitro transcription reaction of any one of the preceding claims, wherein the NTPs comprise unmodified adenosine triphosphate (ATP), unmodified cytidine triphosphate (CTP), unmodified uridine triphosphate (UTP), and/or unmodified guanosine triphosphate (GTP).

10. The in vitro transcription reaction of any one of the preceding claims, wherein the NTPs comprise modified ATP, modified CTP, modified UTP, optionally 1-methylpseudo-UTP, and/or modified GTP.

11. The in vitro transcription reaction of any one of the preceding claims, wherein the reaction mixture comprises an equimolar concentration of NTPs.

12. The in vitro transcription reaction of any one of claims 1-10, wherein the concentration of NTPs is not equimolar.

13. The in vitro transcription reaction of any one of the preceding claims, wherein the initiator oligonucleotide comprises an adenine-guanine (AG) dinucleotide.

14. The in vitro transcription reaction of claim 13, wherein the AG dinucleotide is at a 3′ end of the initiator oligonucleotide.

15. The in vitro transcription reaction of any one of the preceding claims, wherein the initiator oligonucleotide comprises a nucleotide sequence selected from GCAG, GCAAG, GGCAG, GCGAG, and GCAGG.

16. The in vitro transcription reaction of claim 15, wherein the initiator oligonucleotide comprises GCAG.

17. The in vitro transcription reaction of claim 15, wherein the initiator oligonucleotide comprises GCAAG.

18. The in vitro transcription reaction of claim 15, wherein the initiator oligonucleotide comprises GGCAG.

19. The in vitro transcription reaction of claim 15, wherein the initiator oligonucleotide comprises GCGAG.

20. The in vitro transcription reaction of claim 15, wherein the initiator oligonucleotide comprises GCAGG.

21. The in vitro transcription reaction of any one of claims 2-20, wherein the synthetic modification comprises a synthetic carbon chain, optionally an alkyl chain.

22. The in vitro transcription reaction of any one of claims 2-21, wherein the synthetic modification comprises a biotin or streptavidin molecule and/or a polyethylene glycol spacer, optionally a triethylene glycol spacer or tetraethylene glycol spacer.

23. The in vitro transcription reaction of any one of the preceding claims further comprising water, buffer, and/or pyrophosphatase.

24. The in vitro transcription reaction of claim 23, wherein the buffer comprises magnesium ions, optionally wherein the buffer comprises magnesium acetate.

25. The in vitro transcription reaction of claim 24, wherein the buffer comprises 30 mM magnesium ions, 40 mM magnesium ions, or 50 mM magnesium ions.

26. The in vitro transcription reaction of any one of the preceding claims, wherein the RNA polymerase is a T7 RNA polymerase.

27. The in vitro transcription reaction of any one of the preceding claims, wherein the RNA polymerase is a naturally occurring RNA polymerase.

28. The in vitro transcription reaction of any one of the preceding claims further comprising a ribonucleic acid (RNA) encoded by the DNA.

29. A method comprising maintaining the in vitro transcription reaction of any one of claims 1-28 at in vitro transcription conditions to produce a ribonucleic acid (RNA).

30. The method of claim 29, wherein the in vitro transcription conditions include maintaining a temperature of about 37 degrees Celsius for about 30 minutes to about 3 hours.

31. An RNA produced by the method of claim 29 or 30.
